(19) United States
(12) Patent Application Publication
(10) Pub. No.: US 2012/0156133 A1
(43) Pub. Date: Jun. 21, 2012

(54) NON-RADIOACTIVE PHOSPHOLIPID COMPOUNDS, COMPOSITIONS, AND METHODS OF USE

(75) Inventors: Anatoly Pinchuk, Madison, WI (US); Marc Longino, Verona, WI (US); Jamey P. Weichert, Fitchburg, WI (US); William R. Clarke, Colgate, WI (US); Abram M. Vaccaro, Stoughton, WI (US); Irawati Kandela, Madison, WI (US)

(73) Assignee: Cellectar, Inc., Madison, WI (US)

(21) Appl. No.: 13/403,445

(22) Filed: Feb. 23, 2012

Related U.S. Application Data
(63) Continuation of application No. 12/879,167, filed on Sep. 10, 2010.
(60) Provisional application No. 61/241,759, filed on Sep. 11, 2009.
(60) Provisional application No. 61/241,762, filed on Sep. 11, 2009.
(60) Provisional application No. 61/309,213, filed on Mar. 1, 2010.
(60) Provisional application No. 61/309,187, filed on Mar. 1, 2010.

(51) Int. Cl. A61K 51/04; A61P 35/00; A61K 31/685

(52) U.S. Cl. 424/1.77; 514/78

(57) ABSTRACT

The present invention provides phospholipid ether and alkyl phospholipid compounds and their combinations with other cancer therapy agents. More specifically, the invention relates to the use of phospholipid ether compounds comprising a “cold” isotope of iodine, e.g. ¹²⁷I, or H, for treating cancer and combinations of phospholipid compounds comprising radioactive (i.e., “hot”) and non-radioactive (i.e., “cold”) isotopes of iodine.

### 1. FIELD OF THE INVENTION

The present invention generally relates to compositions and methods for treatment of solid cancers.

### 2. BACKGROUND OF THE INVENTION

Phospholipid ether and alkyl phospholipid compounds (referred to generically as “PLE compounds”) comprising radioactive (i.e., “hot”) isotopes of iodine and their use in cancer treatment and diagnosis are known in the art. See, for example, U.S. Pat. No. 6,417,384 B1 and WO 2007/013894 A2. In particular, compound CLR1404 (18-(p-iodophenyl)octadecyl phosphocholine) is known and is currently undergoing clinical trials for treatment of various solid cancers.

However, there is a need to further explore the potential of PLE compounds labeled with a non-radioactive (i.e., “cold”) isotope of iodine. In addition, there is a need for new methods of treating cancer with synergistic compositions comprising PLE compounds and Akt inhibitors.

### 3. SUMMARY OF THE INVENTION

In one embodiment, the invention provides a method of treating a solid cancer comprising administering to a patient in need thereof a therapeutically effective amount of a nonradioactive phospholipid compound selected from:

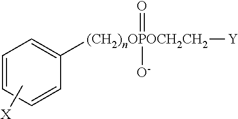

where X is: a) a nonradioactive isotope of iodine or b) H; n is an integer between 12 and 30; and Y is selected from the group consisting of N⁺H₃, HN⁺(R)₂, N⁺H₂R, and N⁺(R)₃, wherein R is an alkyl or arylalkyl substituent and

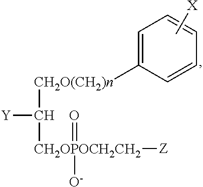

where X is: a) a nonradioactive isotope of iodine or b) H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent.

In a preferred embodiment, the nonradioactive phospholipid compound for use in the methods of the invention is selected from the group consisting of 18-(p-Iodophenyl)octadecyl phosphocholine, 1-O-[8-(p-Iodophenyl)octadecyl]-1,3-propanediol-3-phosphocholine, and 1-O-[8-(p-Iodophenyl)octadecyl]-2-O-methyl-rac-glycero-3-phosphocholine, and pharmaceutically acceptable salts thereof, wherein iodine is a nonradioactive isotope.

In an even more preferred embodiment, the phospholipid compound for use in the methods of the invention is of the formula:

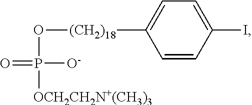

wherein I is a nonradioactive isotope of iodine, or a pharmaceutically acceptable salt thereof. This compound is also referred to as “CLR1401” throughout the application.

In a preferred embodiment, solid cancers are selected from the group consisting of lung cancer, breast cancer, glioma, squamous cell carcinoma, prostate cancer, melanoma, renal cancer, colorectal cancer, ovarian cancer, pancreatic cancer, sarcoma, and stomach cancer.

In another embodiment the invention provides a nonradioactive phospholipid compound selected from:

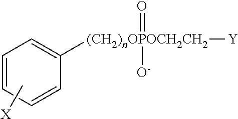

where X is H; n is an integer between 12 and 30; and Y is selected from the group consisting of N⁺H₃, HN⁺(R)₂, N⁺H₂R, and N⁺(R)₃, wherein R is an alkyl or arylalkyl substituent and

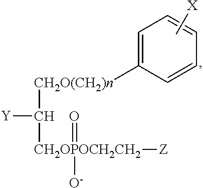

where X is H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent.

In another embodiment, the invention provides a combination pharmaceutical agent for the treatment of solid cancer comprising the non-radioactive phospholipid compounds of the invention and another chemotherapeutic agent.

In a preferred embodiment, the other chemotherapeutic agent comprises a radioactive phospholipid compound selected from:

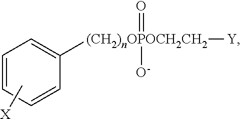

where X is a radioactive isotope of iodine; n is an integer between 12 and 30; and Y is selected from the group consisting of N⁺H₃, HN⁺(R)₂, N⁺H₂R, and N⁺(R)₃, wherein R is an alkyl or arylalkyl substituent or

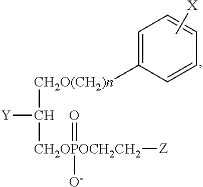

where X is a radioactive isotope of iodine; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent.

In one embodiment, the invention provides a combination pharmaceutical agent comprising: a) a radioactive phospholipid compound selected from

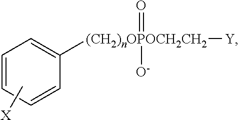

where X is a radioactive isotope of iodine; n is an integer between 12 and 30; and Y is selected from the group consisting of N⁺H₃, HN⁺(R)₂, N⁺H₂R, and N⁺(R)₃, wherein R is an alkyl or arylalkyl substituent or

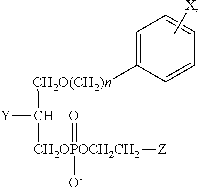

where X is a radioactive isotope of iodine; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent or a pharmaceutically acceptable salt thereof and b) a protein kinase B (Akt) inhibitor.

In a preferred embodiment, said protein kinase B (Akt) inhibitor is a nonradioactive phospholipid compound selected from:

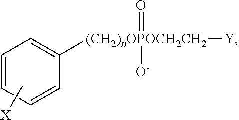

where X is: a) a nonradioactive isotope of iodine or b) H; n is an integer between 12 and 30; and Y is selected from the group consisting of N⁺H₃, HN⁺(R)₂, N⁺H₂R, and N⁺(R)₃, wherein R is an alkyl or arylalkyl substituent and

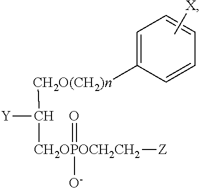

where X is: a) a nonradioactive isotope of iodine or b) H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent, or a pharmaceutically acceptable salt thereof.

In a preferred embodiment, the radioactive isotope of iodine in the radioactive phospholipid compound is selected from the group consisting of ¹²³I, ¹²⁴I, ¹²⁵I, and ¹³¹I; and even more preferably, from the group consisting of ¹²⁵I and ¹³¹I.

In a more preferred embodiment, the radioactive phospholipid compound is selected from the group consisting of 18-(p-Iodophenyl)octadecyl phosphocholine, 1-O-[18-(p-Iodophenyl)octadecyl]-1,3-propanediol-3-phosphocholine, and 1-O-[18-(p-Iodophenyl)octadecyl]-2-O-methyl-rac-glycero-3-phosphocholine, and pharmaceutically acceptable salts thereof, wherein iodine is a radioactive isotope.

In an even more preferred embodiment, the invention provides a combination pharmaceutical agent comprising a nonradioactive phospholipid compound of the formula:

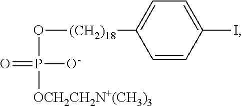

wherein I is a nonradioactive isotope of iodine, and a radioactive phospholipid compound of the formula:

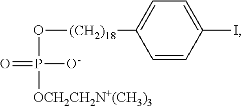

wherein I is a radioactive isotope of iodine.

The invention also provides pharmaceutical compositions comprising the combination agents of the invention.

In one embodiment, a nonradioactive phospholipid compound of the invention and another chemotherapeutic agent (e.g., a radioactive phospholipid compound) are formulated as a single composition.

In a preferred embodiment, CLR1401 (18-(p-Iodophenyl)octadecyl phosphocholine, wherein I is a nonradioactive isotope of iodine) and CLR1404 (18-(p-Iodophenyl)octadecyl phosphocholine, wherein I is a radioactive isotope of iodine) are formulated as a single composition, and the ratio of CLR1401 to CLR1404 is about 10:1 by weight.

In another embodiment, a phospholipid compound of the invention and another chemotherapeutic agent (e.g., a radioactive phospholipid compound) are formulated as separate compositions.

If formulated as separate compositions, the nonradioactive phospholipid compounds (for example, CLR1401) may be administered prior to, or concurrently with, administration of the radioactive phospholipid compounds (for example, CLR1404).

In another embodiment, the invention also provides methods for the treatment of solid cancers comprising administering to a patient in need thereof a therapeutically effective amount of a combination pharmaceutical agent of the invention.

In a preferred embodiment, when the provided combination pharmaceutical agents are administered to a human patient, the serum concentration of the nonradioactive compound may reach between about 5 μM and about 10 μM.

The invention also provides methods of treating a solid cancer comprising administering to a patient in need thereof a therapeutically effective amount of the combination pharmaceutical agents of the invention.

In one embodiment, the therapeutically effective amound of the combination pharmaceutical agent is from about 7 mCi to about 700 mCi.

In a preferred embodiment, the solid cancers are selected from the group consisting of lung cancer, breast cancer, glioma, squamous cell carcinoma, prostate cancer, melanoma, renal cancer, colorectal cancer, ovarian cancer, pancreatic cancer, sarcoma, and stomach cancer.

### 4. BRIEF DESCRIPTION OF THE DRAWINGS

FIG. 1 is an ELISA chart demonstrating dose-dependent decrease in the amount of active Akt (pAkt, S473) levels in A549 cells with increasing doses of ¹²⁷I-CLR1401.

FIG. 2 is an ELISA chart demonstrating dose-dependent decrease in the amount of active Akt (pAkt, S473) levels in PC-3 cells with increasing doses of ¹²⁷I-CLR1401.

FIGS. 3A and 3B demonstrate linearity of percent (%) inhibition of active Akt (pAkt, S437) and concentration of ¹²⁷I-CLR1401 in A549 and PC-3 cells, respectively.

FIG. 4 demonstrates a chart of potential targets of ¹²⁷I-CLR1401 which would cause a decrease in the amount of active Akt (pAkt, S473).

FIG. 5A demonstrates the effect of low doses of ¹²⁷I-CLR1401 on the growth of A549 cells.

FIG. 5B demonstrates the effect of midrange doses of ¹²⁷I-CLR1401 on the growth of A549 cells.

FIG. 5C demonstrates the effect of high doses of ¹²⁷I-CLR1401 on the growth of A549 cells.

FIG. 5D demonstrates dose-dependent decrease in growth in A549 cells treated with ¹²⁷I-CLR1401.

FIG. 6 demonstrates the effect of increasing mass dose of ¹²⁵I-CLR1404 on the uptake and retention of ¹²⁵I-CLR1404 by A549 cells at 24 hours post treatment.

FIG. 7 demonstrates the effect of increasing mass dose of ¹²⁷I-CLR1401 on the uptake and retention of a fixed tracer amount of ¹²⁵I-CLR1404 (0.588 μM) by A549 cells at 24 hours post treatment.

FIG. 8 demonstrates comparison of the effect of increasing mass dose of ¹²⁷I-CLR1401 on the uptake and retention of ¹²⁵I-CLR1404 (0.588 μM) by A549 cells at 24 hours post treatment with control.

FIG. 9A demonstrates a plot of ¹²⁵I-CLR1404 concentration vs. fold increase in uptake and retention.

FIG. 9B demonstrates a plot of a combination of ¹²⁵I-CLR1404 and ¹²⁷I-CLR1401 concentration vs. fold increase in uptake and retention.

FIG. 10 demonstrates a plot of prostate carcinoma (PC-3) growth response to the treatment by combinations of ¹³¹I-CLR1404 and different dosages of ¹²⁷I-CLR1401.

FIG. 11 demonstrates a Kaplan-Meyer plot of % survival of mice injected with PC-3 cells.

FIG. 12 demonstrates a plot of non-small cell lung cancer cells (A549) growth response to the treatment with ¹³¹I-CLR1404, ¹²⁷I-CLR1401, and a combination of ¹³¹I-CLR1404 and ¹²⁷I-CLR1401.

FIG. 13 demonstrates a plot of human mammary gland adenocarcinoma cells (MDA-MB-231) growth response to the treatment with ¹³¹I-CLR1404, ¹²⁷I-CLR1401, and combinations of ¹³¹I-CLR1404 and ¹²⁷I-CLR1401.

FIG. 14 demonstrates a Kaplan-Meyer plot of % survival of mice injected with MDA-MB-231 cells.

FIG. 15 demonstrates a plot of non-small cell lung cancer cells (A549) growth response to the treatment with ¹²⁷I-CLR1401 versus the treatment with erlotinib.

FIG. 16 demonstrates a plot of % survival of mice injected with A549 cells.

### 5. DETAILED DESCRIPTION OF THE INVENTION

### 5.1 Definitions

The term “composition” includes a product comprising the specified ingredients (and in the specified amounts, if indicated), as well as any product which results, directly or indirectly, from combination of the specified ingredients in the specified amounts. By “pharmaceutically acceptable” it is meant the diluent, excipient or carrier must be compatible with the other ingredients of the formulation and not deleterious to the recipient thereof.

The term “administering” or “administration” includes any means for introducing phospholipid compounds of the invention and other therapeutic agents, including radiotherapy and chemotherapy, into the body, preferably into the systemic circulation. Examples include but are not limited to oral, buccal, sublingual, pulmonary, transdermal, transmucosal, as well as subcutaneous, intraperitoneal, intravenous, and intramuscular injection.

The term “therapeutically effective amount” means an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors.

The term “treating” has a commonly understood meaning of administration of a remedy to a patient who has or is suspected of having a disease or a condition. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

The term “contacting” means that the phospholipid compound or the combination pharmaceutical agent used in the present invention is introduced into a patient receiving treatment, and the compound is allowed to come in contact in vivo.

The terms “phospholipid ether compound” and “phospholipid compound” are used interchangeably for the purposes of the present application.

The term “CLR1401” means the compound of the formula:

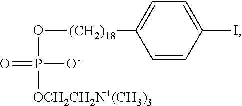

wherein I is a nonradioactive isotope of iodine, or a pharmaceutically acceptable salt thereof.

The term “CLR1404” means the compound of the formula:

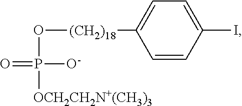

wherein I is a radioactive isotope of iodine, or a pharmaceutically acceptable salt thereof.

The term “crystalline forms” and related terms herein refers to the various crystalline modifications of a given substance, including, but not limited to, polymorphs, solvates, hydrates, co-crystals and other molecular complexes, as well as salts, solvates of salts, hydrates of salts, other molecular complexes of salts, and polymorphs thereof.

The compounds of the invention encompass pharmaceutically acceptable salts of the phosphocholine portion of the compounds. The compounds of the invention are also preferably inner salts (zwitterions) themselves.

The term “pharmaceutically acceptable salts” is meant to include salts of active compounds which are prepared with relatively nontoxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic; propionic; isobutyric; maleic; malonic; benzoic; succinic; suberic; fumaric; mandelic; phthalic; benzenesulfonic; toluenesulfonic, including p-toluenesulfonic, m-toluenesulfonic, and o-toluenesulfonic; citric; tartaric; methanesulfonic; and the like. Also included are salts of amino acids such as arginate and the like, and salts of organic acids like glucuronic or galactunoric acids and the like (see, for example, Berge et al. J. Pharm. Sci. 66:1-19 (1977)).

As used herein, a salt or polymorph that is “pure,” i.e., substantially free of other polymorphs, contains less than about 10% of one or more other polymorphs, preferably less than about 5% of one or more other polymorphs, more preferably less than about 3% of one or more other polymorphs, most preferably less than about 1% of one or more other polymorphs.

The terms, “polymorphs” and “polymorphic forms” and related terms herein refer to crystal forms of a molecule. Different polymorphs may have different physical properties such as, for example, melting temperatures, heats of fusion, solubilities, dissolution rates and/or vibrational spectra as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

The term “alkyl,” as used herein refers to monovalent saturated aliphatic hydrocarbon groups, particularly, having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyl” as defined below.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.”

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

The term “subject” is defined herein to include animals such as mammals, including, but not limited to, primates (e.g., humans, monkeys, apes), cows, sheep, goats, horses, dogs, cats, rabbits, rats, mice and the like. In preferred embodiments, the subject is a human.

As used herein, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### 5.2 Methods for Treatment of a Solid Cancer Utilizing Nonradioactive Phospholipid Compounds

In one embodiment, the invention provides a method of treating a solid cancer comprising administering to a patient in need thereof a therapeutically effective amount of a nonradioactive phospholipid compound selected from:

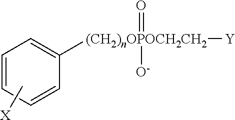

where X is: a) a nonradioactive isotope of iodine or b) H; n is an integer between 12 and 30; and Y is selected from the group comprising N⁺H₃, HN⁺(R)₂, N⁺H₂R, and N⁺(R)₃, wherein R is an alkyl or arylalkyl substituent and

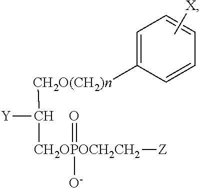

where X is: a) a nonradioactive isotope of iodine or b) H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent, or a pharmaceutically acceptable salt thereof.

In a preferred embodiment, the nonradioactive phospholipid compound for use in the methods of the invention is selected from the group consisting of 18-(p-Iodophenyl)octadecyl phosphocholine, 1-O-[8-(p-Iodophenyl)octadecyl]-1,3-propanediol-3-phosphocholine, and 1-O-[8-(p-Iodophenyl)octadecyl]-2-O-methyl-rac-glycero-3-phosphocholine, and pharmaceutically acceptable salts thereof, wherein iodine is a nonradioactive isotope.

In an even more preferred embodiment, the phospholipid compound for use in the methods of the invention is of the formula:

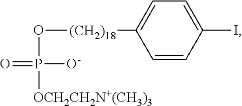

wherein I is a nonradioactive isotope of iodine, or a pharmaceutically acceptable salt thereof. This compound is also referred to as “CLR1401” throughout the application.

The non-radioactive phospholipid compounds, wherein I is a nonradioactive isotope of iodine (e.g., ¹²⁷I) can be made by methods similar to those used to make the radioactive versions of these compounds, described, for example, in Synthesis and Structure-Activity Relationship Effects on the Tumor Avidity of Radioiodinated Phospholipid Ether Analogues, Pinchuk et al, J. Med. Chem. 2006, 49, 2155-2165.

The solid cancers that can be treated with the compounds of the present invention include, but are not limited to, lung cancer, breast cancer, glioma, squamous cell carcinoma, prostate cancer, melanoma, renal cancer, colorectal cancer, ovarian cancer, pancreatic cancer, sarcoma, and stomach cancer.

It is to be understood that the compounds and methods of the present invention encompass the compounds in any racemic, optically-active, polymorphic, or stereoisomeric forms, or mixtures thereof. In one embodiment, the phospholipid compounds may include pure (R)-isomers. In another embodiment, the phospholipid compounds may include pure (S)-isomers. In another embodiment, the phospholipid compounds may include a mixture of the (R) and the (S) isomers. In another embodiment, the phospholipid compounds may include a racemic mixture comprising both (R) and (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The compounds suitable for use in the present invention can exist in unsolvated as well as solvated forms, including hydrated forms, e.g., hemi-hydrate. In general, the solvated forms, with pharmaceutically acceptable solvents such as water, ethanol, and the like are equivalent to the unsolvated forms for the purposes of the invention.

Certain compounds of the invention also form pharmaceutically acceptable salts, e.g., acid addition salts. For example, the nitrogen atoms may form salts with acids. Examples of suitable acids for salt formation are hydrochloric, sulfuric, phosphoric, acetic, citric, oxalic, malonic, salicylic, malic, furmaric, succinic, ascorbic, maleic, methanesulfonic and other mineral carboxylic acids well known to those in the art. The salts are prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et al., “Pharmaceutical Salts,” J. Pharm. Sci., 66:1-19 (1977).

Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulfonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts, alkaline earth metal salts, e.g. calcium or magnesium salts; and salts formed with suitable organic ligands, e.g. quaternary ammonium salts.

The compounds of the present invention can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

Where the compounds according to the invention have at least one asymmetric center, they may accordingly exist as enantiomers. Where the compounds according to the invention possess two or more asymmetric centers, they may additionally exist as diastereoisomers. It is to be understood that all such isomers and mixtures thereof in any proportion are encompassed within the scope of the present invention.

### 5.3 Compositions of Nonradioactive Phospholipid Compounds and Other Chemotherapeutic Agents

In another embodiment the invention provides a nonradioactive phospholipid compound selected from:

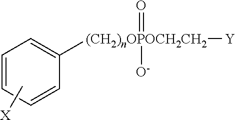

where X is H; n is an integer between 12 and 30; and Y is selected from the group consisting of N⁺H₃, HN⁺(R)₂, N⁺H₂R, and N⁺(R)₃, wherein R is an alkyl or arylalkyl substituent and

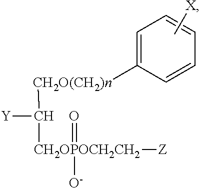

where X is H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent.

In another embodiment, the invention provides a combination pharmaceutical agent for the treatment of a solid cancer comprising the non-radioactive phospholipid compounds and another chemotherapeutic agent.

While not wishing to be bound to any specific theory, it is currently believed, based on the conducted experiments, that nonradioactive phospholipid compounds are able to inhibit or block activation of one of the key signaling and survival enzymes, Akt. (Also known as protein kinase B). Therefore, it is believed that combinations of the nonradioactive phospholipid compounds with other chemotherapeutic agents will have a synergistic effect on the treatment of solid cancers.

As is shown in the Examples, there is a dose response relationship between the addition of CLR1401 (one of the currently described nonradioactive compounds) and the inhibition of Akt.

In one embodiment, the other chemotherapeutic agent that can be synergistically used in the combinations of the present invention is a radioactive phospholipid compound selected from:

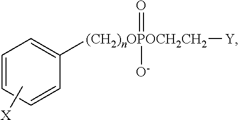

where X is a radioactive isotope of iodine; n is an integer between 12 and 30; and Y is selected from the group consisting of N⁺H₂, HN⁺(R)₂, N⁺H₂R, and N⁺(R)₃, wherein R is an alkyl or arylalkyl substituent or

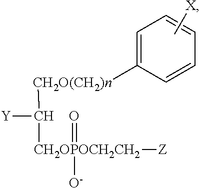

where X is a radioactive isotope of iodine; n is an integer between 12 and 30; Y is selected from the group consisting of H, OH, COOH, COOR and OR, and Z is selected from the group consisting of N⁺H₂, HN⁺(R)₂, N⁺H₂R, and N⁺(R)₃, wherein R is an alkyl or arylalkyl substituent, or a pharmaceutically acceptable salt thereof.

The radioactive phospholipid compounds are known and described, for example, in U.S. Pat. No. 6,417,384 B1.

In a preferred embodiment, the invention provides a combination pharmaceutical agent comprising: a) a radioactive phospholipid compound selected from:

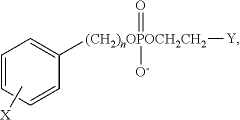

where X is a radioactive isotope of iodine; n is an integer between 12 and 30; and Y is selected from the group consisting of N⁺H₂, HN⁺(R)₂, N⁺H₂R, and N⁺(R)₃, wherein R is an alkyl or arylalkyl substituent or

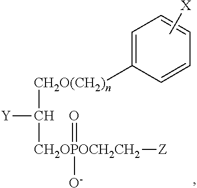

where X is a radioactive isotope of iodine; n is an integer between 12 and 30; Y is selected from the group consisting of H, OH, COOH, COOR and OR, and Z is selected from the group consisting of N⁺H₂, HN⁺(R)₂, N⁺H₂R, and N⁺(R)₃, wherein R is an alkyl or arylalkyl substituent, or a pharmaceutically acceptable salt thereof and b) a protein kinase B (Akt) inhibitor.

In a preferred embodiment, the protein kinase B (Akt) inhibitor is a nonradioactive phospholipid compound selected from:

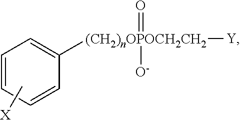

where X is: a) a nonradioactive isotope of iodine or b) H; n is an integer between 12 and 30; and Y is selected from the group consisting of N⁺H₃, HN⁺(R)₂, N⁺H₂R, and N⁺(R)₃, wherein R is an alkyl or arylalkyl substituent and

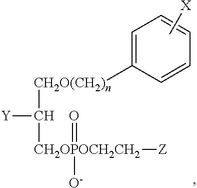

where X is: a) a nonradioactive isotope of iodine or b) H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent, or a pharmaceutically acceptable salt thereof.

In a preferred embodiment, the radioactive isotope of iodine in the radioactive phospholipid compound is selected from the group consisting of ¹²³I, ¹²⁴I, ¹²⁵I, and ¹³¹I; and even more preferably, from the group consisting of ¹²⁵I and ¹³¹I.

In a more preferred embodiment, the radioactive phospholipid compound is selected from the group consisting of 18-(p-Iodophenyl)octadecyl phosphocholine, 1-O-[18-(p-Iodophenyl)octadecyl]-1,3-propanediol-3-phosphocholine, and 1-O-[8-(p-Iodophenyl)octadecyl]-2-O-methyl-rac-glycero-3-phosphocholine, wherein iodine is a radioactive isotope.

In an even more preferred embodiment, the invention provides a combination pharmaceutical agent comprising: a) CLR1401, which is a nonradioactive phospholipid compound of the formula:

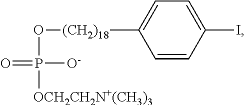

wherein I is a nonradioactive isotope of iodine, and b) CLR1404, which is a radioactive phospholipid compound of the formula:

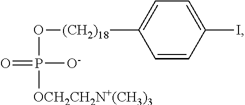

wherein I is a radioactive isotope of iodine.

### 5.4 Pharmaceutical Compositions

Compositions of the present invention may be prepared as a single unit dose or as a plurality of single unit doses. As used herein, a “unit dose” means a discrete amount of the composition comprising a predetermined amount of the active ingredient. The amount of the active ingredient is generally equal to the dosage of the active ingredient that would be administered to a patient or a fraction thereof.

Compositions of the present invention may be liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20™, Tween 80™, Pluronic F68™, bile acid salts), solubilizing agents (e.g., glycerol, polyethylene glycerol), anti-oxidants (e.g., ascorbic acid, sodium metabisulfite), preservatives (e.g., Thimerosal™, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

In a preferred embodiment, compositions of the present invention comprise a compound of the present invention, polysorbate, ethanol, and saline.

Also encompassed by the invention are methods of administering particulate compositions coated with polymers (e.g., poloxamers or poloxamines). Other embodiments of the compositions incorporate particulate forms protective coatings, protease inhibitors or permeation enhancers for various routes of administration, including topical, parenteral, pulmonary, nasal and oral. In some embodiments, the pharmaceutical composition is administered parenterally, paracancerally, transmucosally, tansdermally, intramuscularly, intravenously, intradermally, subcutaneously, intraperitonealy, intraventricularly, intracranially and intratumorally.

Further, as used herein “pharmaceutically acceptable carriers” are well known to those skilled in the art and include, but are not limited to, 0.01-0.1 M and preferably 0.05M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringers dextrose, and the like. Preservatives and other additives may also be present, such as, for example, antimicrobials, antioxidants, collating agents, inert gases and the like.

Controlled or sustained release compositions according to the invention include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors. Other embodiments of the compositions according to the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparation can comprise the phospholipid compound alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the phospholipid compound can be administered to a patient by, for example, subcutaneous implantation of a pellet. In a further embodiment, a pellet provides for controlled release of tumor-specific phospholipid ether analog over a period of time. The preparation can also be administered by intravenous, intraarterial, or intramuscular injection of a liquid preparation oral administration of a liquid or solid preparation, or by topical application. Administration can also be accomplished by use of a rectal suppository or a urethral suppository.

The pharmaceutical preparations administrable by the invention can be prepared by known dissolving, mixing, granulating, or tablet-forming processes. For oral administration, the tumor-specific phospholipid ether analogs or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intra-arterial, or intramuscular injection), the tumor-specific phospholipid ether analogs or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Such compositions may be prepared as injectables, either as liquid solutions or suspensions; however, solid forms suitable for solution in, or suspension in, liquid prior to injection can also be prepared. The preparation can also be emulsified. Active therapeutic ingredients are often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

In one embodiment, a pharmaceutical composition comprises a nonradioactive phospholipid compound of the present invention or a pharmaceutically acceptable salt thereof.

In another embodiment, the invention provides a combination pharmaceutical agent for the treatment of a solid cancer comprising a nonradioactive phospholipid compound of the invention or a pharmaceutically acceptable salt thereof and another chemotherapeutic agent, wherein said combination pharmaceutical agent is formulated as a single composition.

In another embodiment, the invention provides a combination pharmaceutical agent for the treatment of a solid cancer comprising a nonradioactive phospholipid compound of the invention or a pharmaceutically acceptable salt thereof and another chemotherapeutic agent, wherein said combination pharmaceutical agent is formulated as separate compositions.

In a preferred embodiment, the invention provides a combination pharmaceutical agent for the treatment of a solid cancer comprising: a) a ¹²⁷I-CLR1401 (also referred to as I-127-CLR1401) or a pharmaceutically acceptable salt thereof and b) ¹³¹I-CLR1404 (also referred to as I-131-CLR1404) or ¹²⁵I-CLR1404 (also referred to as I-125-CLR1404), wherein said combination pharmaceutical agent is formulated as a single composition.

In an even more preferred embodiment, the invention provides a combination pharmaceutical agent for the treatment of a solid cancer comprising: a) a ¹²⁷I-CLR1401 or a pharmaceutically acceptable salt thereof and b) ¹³¹I-CLR1404 or ¹²⁵I-CLR1404, wherein said combination pharmaceutical agent is formulated as a single composition, and wherein the ratio of ¹²⁷I-CLR1401 to ¹³¹I-CLR1404 or ¹²⁵I-CLR1404 is about 10:1 by weight.

If formulated as separate compositions, the nonradioactive phospholipid compounds (for example, CLR1401) may be administered prior to, or concurrently with, administration of the radioactive phospholipid compounds (for example, CLR1404).

### 5.5 Methods for the Treatment of Solid Cancers Utilizing Combination Pharmaceutical Agents

In another embodiment, the invention also provides methods for the treatment of solid cancers comprising administering to a patient in need thereof a therapeutically effective amount of a combination pharmaceutical agent of the invention.

In a preferred embodiment, the solid cancers are selected from the group consisting of lung cancer, breast cancer, glioma, squamous cell carcinoma, prostate cancer, melanoma, renal cancer, colorectal cancer, ovarian cancer, pancreatic cancer, sarcoma, and stomach cancer.

The compounds and pharmaceutical compositions of the present invention may be administered either as a one-time administration or over the time course of days, weeks, months, or even years.

In a preferred embodiment, when the provided combination pharmaceutical agents are administered to a human patient, the serum concentration of the nonradioactive compound may reach between about 5 μM and about 10 μM.

The nonradioactive phospholipid compounds (for example, CLR1401) may be administered prior to, concurrently with, or after administration of the radioactive phospholipid compounds (for example, CLR1404).

Generally, the therapeutically effective amount of the combination pharmaceutical agents in humans is preferably between 0.21-21 mg (equivalent to a 7-700 mCi, total mass dose range) and between 0.03-0.21 mg/kg (equivalent to 1-7 mCi/kg, by weight dose range).

Preferably, the effective radioactive dose (i.e. total mass dose range) is generally between 7-700 mCi; more preferably, between 10-500 mCi; more preferably, 50-250 mCi; and most preferably 80-100 mCi.

The determination of specific dosages and amounts of the phospholipid compounds of the present invention and/or other active ingredients is well within a skill in the art.

### EXAMPLES

### 5.6 Example 1

### The Inhibition of Akt Activation by ¹²⁷I-CLR1401 in Non-Small Cell Lung Cancer and Prostate Adenocarcinoma Cell Lines

A549 cells, obtained from American Type Culture Collection (ATCC), are a human non-small cell lung cancer cell line. A549 cells have wild-type functional PTEN.

PC-3 cells, obtained from American Type Culture Collection (ATCC), are a human prostate carcinoma cell line. PC-3 cells have a homozygous deletion of PTEN.

### Experimental Methods

A549 and PC-3 cells were plated at a density of 200,000 cells per ml.

All treatments were preformed in triplicate.

A549 and PC-3 cells are treated with ¹²⁷I-CLR1401 for 24 hours.
0, 3, 5, 10 μM of ¹²⁷I-CLR1401
Protein was isolated from treated cells.
The level of activated Akt was determined by examining the phosphorylated (active) form at Ser473 by enzyme linked immunosorbent assay (ELISA).
   PathScan Phospho-Akt1 (Ser473) Sandwich ELISA Kit (Cell Signaling #7160).
ELISA controls:
   Negative Control: A549 and PC-3 cells treated with 50 μM LY294,002 for 24 hrs. LY294,002 is a specific cell permeable phosphatidylinositol 3-kinase (PI3K) inhibitor that inhibits the activation of Akt by affecting the amount of phosphatidylinositol(3,4,5) trisphosphate produced by PI3K.
   Positive Control: A549 and PC-3 cells stimulated with 10 μg/ml Insulin for 24 hrs.
   Negative Control: A549 in serum free media.
1) The ELISA was preformed per the manufacturer's instructions. Briefly, 100 μg of protein from the cell lysates were incubated in the pre-coated 96-well plate overnight at 4° C. The wells were then washed 4 times with 1× Wash Buffer (also provided). Then incubated with the primary Akt antibody for 2 hours at 37° C. with 5% CO₂ in air. After the primary incubation the plate was then washed 4 times with 1× Wash Buffer and incubated with the HRP-linked secondary antibody for 1 hour at 37° C. with 5% CO₂ in air. The plate was then washed 4 times with 1× Wash Buffer and developed using the TMB substrate provided. TMB substrate (tetramethylbenzidine) is a colormetric substrate used in the ELISA assay. TMB (3,3″,5,5″-tetramethylbenzidine) is a chromogen that yields a blue color when oxidized with hydrogen peroxide (catalyzed by HRP) with major absorbances at 370 nm and 652 nm. The color then changes to yellow with the addition of sulfuric or phosphoric acid with maximum absorbance at 450 nm. It is used for the detection of target proteins that have been bound to an antibody that contains a Horseradish Peroxidase tag.
After the 10 minute incubation at 37° C. the reaction was stopped by the addition of stop buffer (also provided). The absorbance was then measured at 450 nm using a Synergy HT microplate reader (BioTek). Data are reported as absorbance at 450 nm.

### 2. Cell Growth Inhibition by ¹²⁷I-CLR1401

1) Growth inhibition induced by ¹²⁷I-CLR1401 was determined by MTT(3-(4,5-dimethylthiazol-2-yl)-2,5-diphenyl tetrasodium bromide) assay. MTT is a pale yellow substrate that is cleaved by living cells to yield a dark blue formazan product. This process requires active mitochondria, and even freshly dead cells do not cleave significant amounts of MTT.

Briefly, A549 cells were plated at a density of 200,000 cells per well in a six well plate and allowed to adhere overnight.

The cells were then treated with varying concentrations of ¹²⁷I-CLR1401 (0.0078, 0.392, 0.784, 1.568, 3.137, 4.705, 7.84, 39.2, 78.4 μM) in triplicate and then collected for the MTT assay at the indicated time points. Cells were then incubated with 0.5 mg/ml MTT in 1×PBS for 3 hours at 37° C. with 5% CO₂ in air.

Absorbance was measured at 540 nm using a Synergy HT microplate reader. The absorbance value at 540 nm is directly proportional to the number of viable cells present.

### Results:

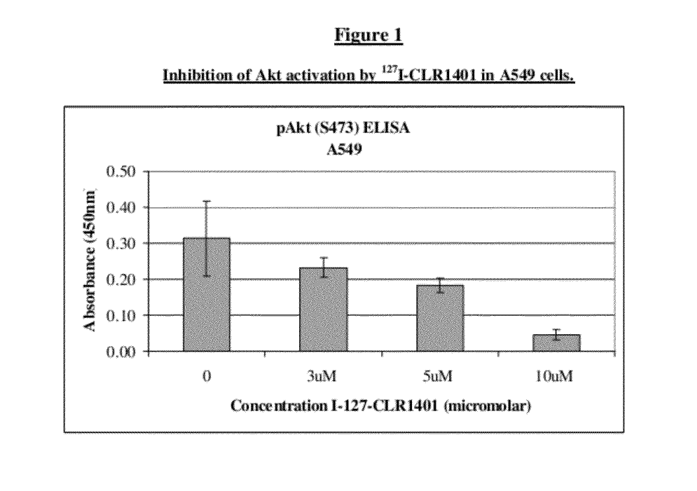

FIG. 1 demonstrates that ¹²⁷I-CLR1404 inhibited activation of Akt in A549 cells. There is a dose-dependent decrease in the amount of active Akt (pAkt, S473) levels with increasing doses of ¹²⁷I-CLR1401.

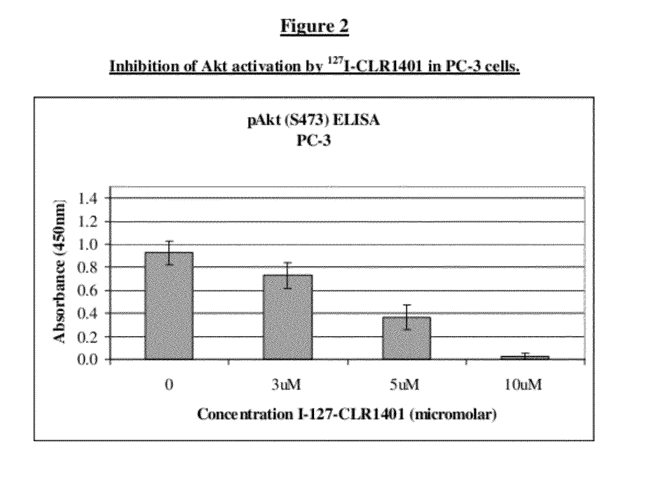

FIG. 2 demonstrates that ¹²⁷I-CLR1404 inhibited activation of Akt in PC-3 cells. There is a dose-dependent decrease in the amount of active Akt (pAkt, S473) levels with increasing doses of ¹²⁷I-CLR1401.

Table 1 demonstrates the percent inhibition of Akt based on decreased levels of phosphorylated Akt at the S473 (serine 473) site. The numbers are taken from the ELISA data demonstrated in FIG. 1 and FIG. 2.

TABLE 1
Percent Inhibition of Akt by ¹²⁷I-CLR1401

| Concentration (μM) | A549 | PC-3 |
|---|---|---|
| 0 | 0% | 0% |
| 3 | 26% | 21% |
| 5 | 42% | 61% |
| 10 | 85% | 97% |

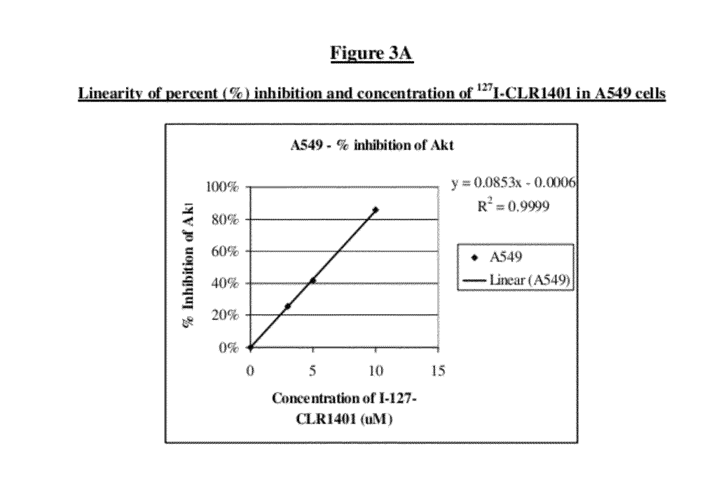

FIGS. 3A and 3B demonstrate linearity of percent (%) inhibition and concentration of ¹²⁷I-CLR1401 in both A549 cells (FIG. 3A) and PC-3 cells (FIG. 3B). There is a linear relationship between Akt inhibition and the used concentration of ¹²⁷I-CLR1401. The IC₅₀ for Akt inhibition is 5.9 μM and 5.0 μM in A549 cells and PC-3 cells, respectively.

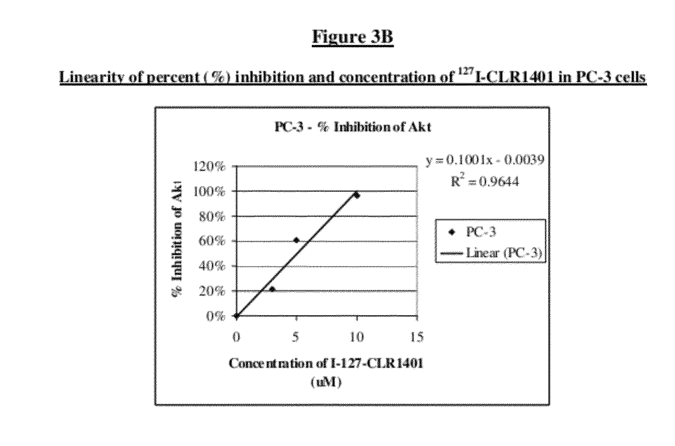

FIG. 4 demonstrates a chart of potential targets for ¹²⁷I-CLR1401 which would cause a decrease in the amount of pAkt (S473).

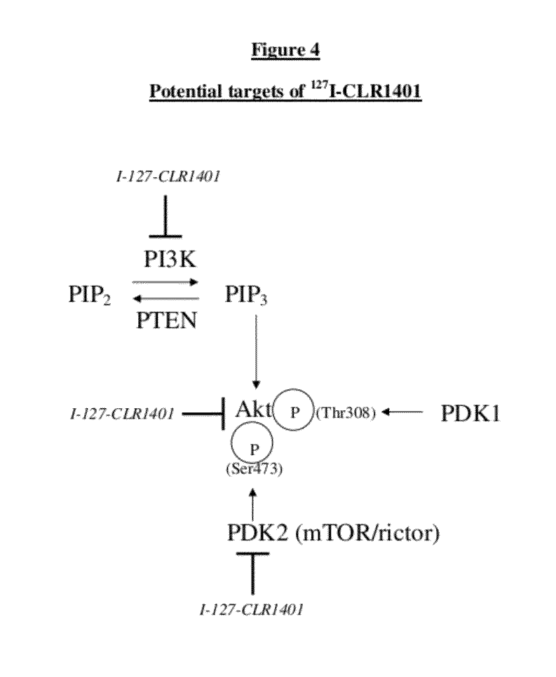

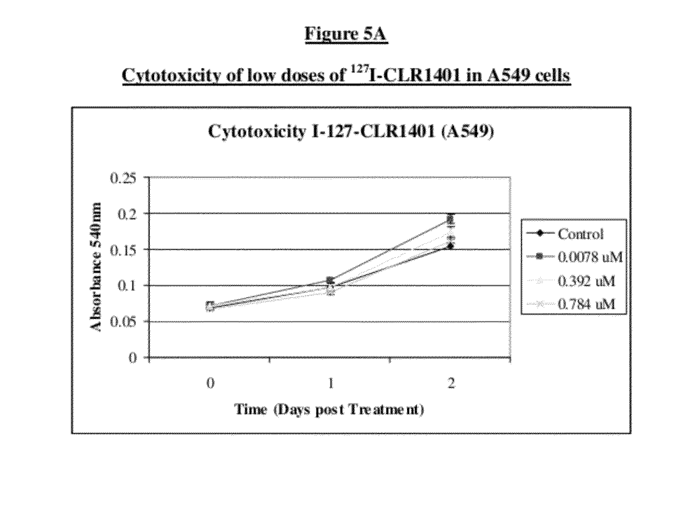

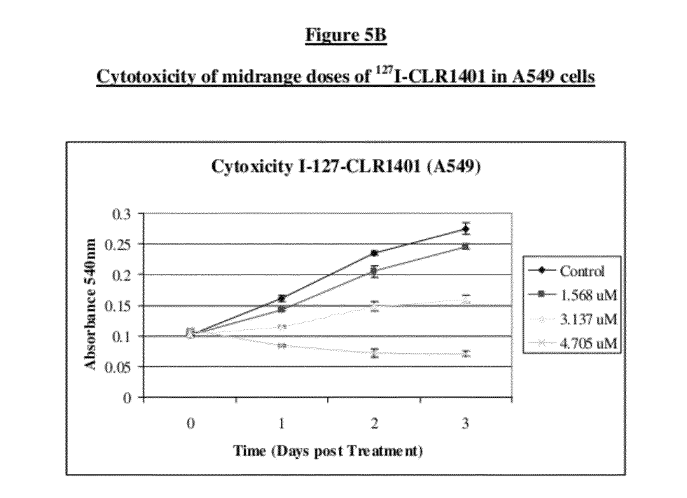

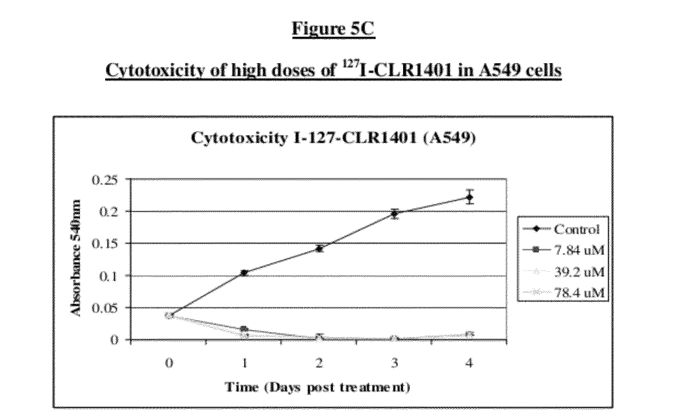

FIG. 5A demonstrates the effect of low doses of ¹²⁷I-CLR1401 on the growth of A549 cells. There was no significant effect. Growth was determined using the MTT assay at the days indicated.

FIG. 5B demonstrates the effect of midrange doses of ¹²⁷I-CLR1401 on the growth of A549 cells. There was a statistically significant, dose-dependent effect. Growth was determined using the MTT assay at the days indicated.

FIG. 5C demonstrates the effect of high doses of ¹²⁷I-CLR1401 on the growth of A549 cells. There was a statistically significant, dose-dependent effect. There was extensive cell death, presumably through apoptosis as seen by observation of membrane blebbing. Growth was determined using the MTT assay at the days indicated.

FIG. 5D demonstrates dose-dependent decrease in growth in A549 cells treated with ¹²⁷I-CLR1401. There was a statistically significant, dose-dependent effect. There was extensive cell death, presumably through apoptosis as seen by observation of membrane blebbing. Growth was determined using the MTT assay at the days indicated. All experiments were performed in triplicate in serum free media.

The fact that the 50% inhibitory concentration of ¹²⁷I-CLR1401 for Akt (5.9 μM) is close to the 50% inhibitory concentration for cell growth (4.5 μM), is strong evidence that the inhibition of Akt is closely linked to the cell death observed. The inhibition of Akt seen in this study is of the basal level of active Akt. In future experiments, it would be important to determine if ¹²⁷I-CLR1401 also inhibits Akt after growth factor stimulation (i.e. insulin stimulation).

There are many potential targets of ¹²⁷I-CLR1401 that would decrease the level of active (phosphorylated) Akt (FIG. 4). The most likely candidates are; PDK2 (mTOR/rictor complex), PI3K, or Akt itself. Because ¹²⁷I-CLR1401 inhibits Akt in PC-3 cells as well as A549, it can be concluded that the inhibition of Akt by ¹²⁷I-CLR1401 is done in a PTEN independent manner. This is due to the fact that PC-3 cells contain a homozygous deletion for the PTEN gene, and therefore do not express any form of the PTEN protein. This information is of particular importance because most cancer types contain inactive or mutated PTEN.

The ability of ¹²⁷I-CLR1401 to inhibit Akt is extremely important when considered as a combination treatment to enhance radiation or traditional chemotherapeutics. Active Akt leads to the degradation of p53 in an MDM2 dependent manner which leads to an increased survival response to radiation. MDM2 (murine double minute 2 protein) is an oncogene. It is an E3 ubiquitin ligase that regulates the level of p53 protein by tagging it with ubiquitin that in turns cause p53 to be shuttled out of the nucleus and into the cytoplasm where it is degraded by proteasomes. Also, active Akt inhibits many known cell cycle inhibitors (e.g. p21 and p27) which would allow cells to continue to proliferate even in the presence of cell cycle arrest signals from traditional chemotherapeutics. By inhibiting Akt with 1271-CLR1401 there exists a strong possibility of synergistic effects when used in combination with traditional radiation and/or chemotherapeutic regimens.

### 5.7 Example 2

### Increasing Concentrations of ¹²⁷I-CLR1401 Increases Uptake and Retention of CLR1404 in A549 Cell Line

Background:

A549 cells are a human non-small cell lung cancer (NSCLC) cell line received from American Type Culture Collection (ATCC).

Treatment of A549 cells with ¹²⁷I-CLR1401 inhibits the activation of protein Akt with an 1050 of 5.9 μM.

Experimental Conditions:

The Uptake and Retention Assay was preformed as described previously. Briefly, A549 human NSCLC cells were plated at a density of 150,000 cells/ml in 6-well plates.

Cells were then allowed to adhere overnight. All plates were then treated with the indicated mass of either ¹²⁵I-CLR1404 with or without ¹²⁷I-CLR1401 as indicated by the experimental parameters.

Cells were incubated in the presence of drug for 24 hrs prior to collection. At 24 hrs post treatment, the media was removed and the cells were washed once with 1 ml of ice cold 1×PBS+1% BSA. The cells were then removed from the plate by trypsinization with 1 ml of 1× Trypsin 1×PBS solution and split into 2 samples of 500 μl each. Both sets of sample were pelleted by centrifugation for 30 seconds at 2000×g at room temperature. The supernatant was removed and discarded.

One pellet for each sample set was resuspended in 200 μl of 1×PBS (Tube 1, Sample 1), and the other pellet was resuspened in 100% EtOH (Tube 2, Sample 1). A 100 μl sample from Tube 1 was taken for evaluation of DNA content in order to determine cell number (data was generated using absorbance at 260 nm in a microplate reader). A 10 μl sample from Tube 2 was taken for evaluation of radioactivity content using a gamma counter. From this data, the activity per cell was determined in triplicate for each treatment. All treatments were preformed in Serum Free media.

The first experiment was performed using only ¹²⁵I-CLR1404 at the mass doses indicated (0.588, 0.980, 1.372, 2.156 μM). Treatments were preformed in triplicate. Data was generated as activity per cell as described above.

All treatments were performed in serum free media.

The second experiment was performed using both ¹²⁵I-CLR1404 and ¹²⁷I-CLR1401.

Treatments were preformed in triplicate.

All treatment groups were given the same tracer amount of ¹²⁵I-CLR1404 (0.5880 μM), then given increasing doses of ¹²⁷I-CLR1404 to achieve a total mass dose as indicated (0.588, 0.980, 1.372, 2.1560 μM). Data was generated as activity per cell as described above. In analysis, the amount of ¹²⁵I-CLR1404 was handled as a tracer and the ratio of concentrations was used to correct for this fact.

All experiments were preformed in serum free media.

Results:

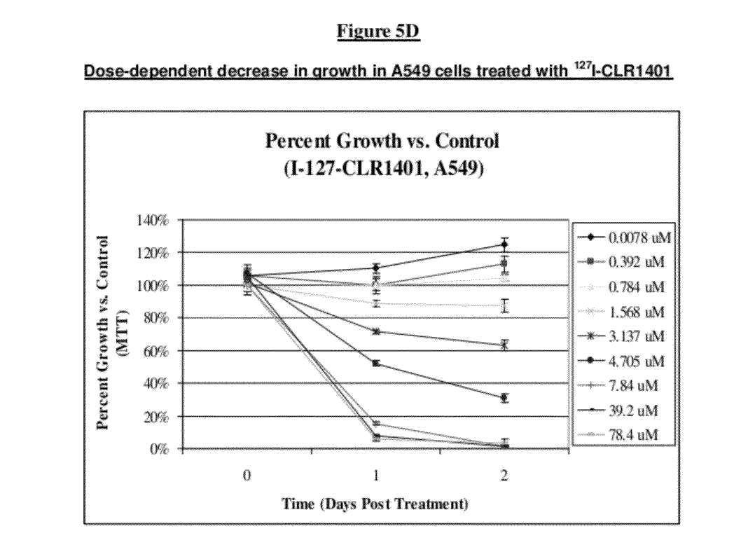

FIG. 6 demonstrates the effect of increasing mass dose of ¹²⁵I-CLR1404 on the uptake and retention of ¹²⁵I-CLR1404 by A549 cells at 24 hours. There is a statistically significant difference between each individual treatment group and the control (0.588 μM) p<0.001.

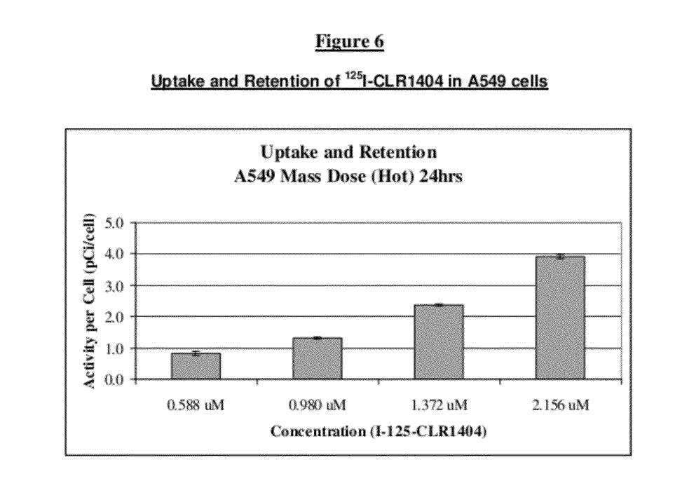

FIG. 7 demonstrates the effect of increasing mass dose of ¹²⁷I-CLR1404 on the uptake and retention of a fixed tracer amount of ¹²⁵I-CLR1404 (0.588 μM) in A549 cells at 24 hours post treatment. There is a statistically significant difference between the 1.372 μM and the 2.156 μM vs. the control (0.588 μM) group the p-values are 0.034 and <0.001 respectively.

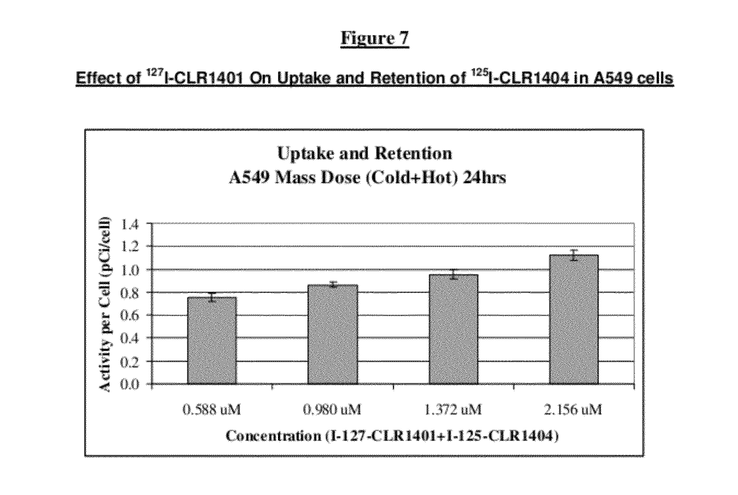

FIG. 8 demonstrates comparison of the effect of increasing Total Mass Dose on the uptake and retention of ¹²⁵I-CLR1401 in A549 cells at 24 hours post treatment. Data is reported as a ratio versus Control (0.588 μM). The ¹²⁷I-CLR1401+¹²⁵I-CLR1404 data is corrected to account for the tracer amount of 1251-CLR1404 added in the presence of increasing concentrations of ¹²⁷I-CLR1401. Concentration values given in the x-axis represent Total Mass Dose pre treatment as a combination of ¹²⁵I-CLR1404+¹²⁷I-CLR1401 treatments.

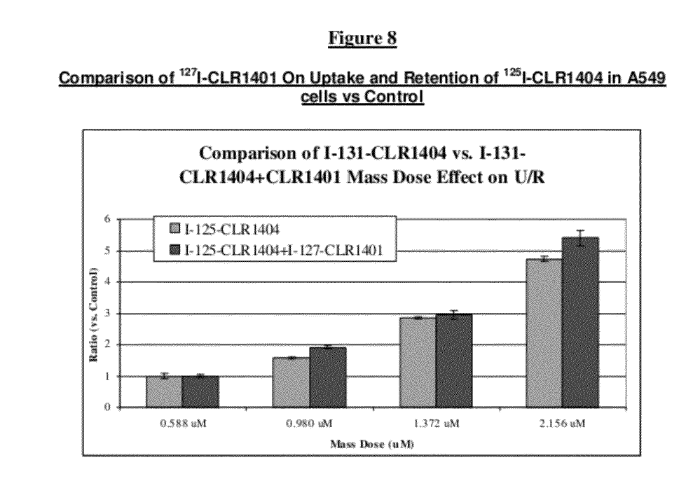

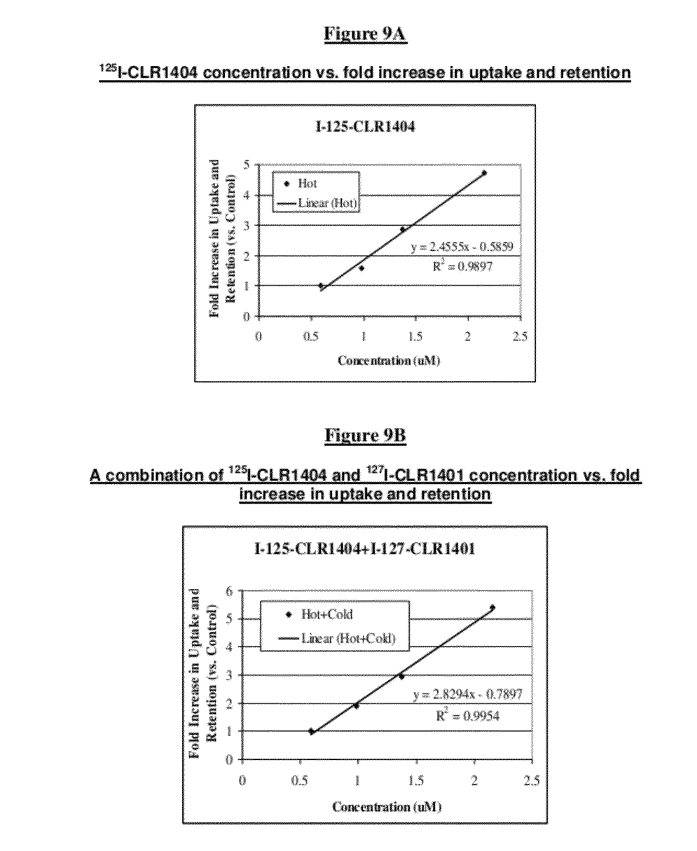

FIGS. 9A and 9B demonstrate a linear relationship between the Total Mass Dose (¹²⁵I-CLR1404 or ¹²⁵I-CLR1404+¹²⁷I-CLR1401) and the fold increase in uptake and retention seen in A549 cells at 24 hours post treatment.

The mechanism by which CRL1404/CLR1401 gains entry and is selectively retained inside of malignant cells is of great interest. By gaining a better understanding as to why CLR1404 is selectively retained we can begin to take greater advantage of the cellular machinery involved. Based on the experiments presented in this report, there is a direct correlation between the amount of CLR1404 present in the system and the amount of CLR1404 that is taken up and retained by A549 cells at 24 hours post treatment. When an increasing mass dose of ¹²⁵I-CLR1404 is given to A549 cells there is a distinct increase in the amount of compound taken up and retained (FIG. 6). This trend is also seen when only a tracer amount of ¹²⁶I-CLR1404 is used and the remaining mass is supplemented with ¹²⁷I-CLR1401 (FIG. 7). By correcting for the tracer amount of ¹²⁶I-CLR1404 given in the second experiment, the fold increase in both experiments is remarkably identical (FIG. 8).

There is a linear relationship between the Total Mass Dose of CLR1404/CLR1401 and the fold increase in uptake seen in A549 cells treated for 24 hours (FIGS. 9A and 9B). The R2 values for the ¹²⁶I-CLR1404 alone and the ¹²⁷I-CLR1401 curves are 0.9897 and 0.9954 respectively (FIGS. 9A and 9B).

### 5.8 Example 3

### ¹²⁷I-CLR1401 Increases the Effectiveness of ¹³¹I-CLR1404 in Treatment of Prostate Cancer

Experimental Conditions:

The PC-3 cell line (human prostate carcinoma) was purchased from American Type Culture Collection (ATCC, Rockville, Md.) and maintained in F-12K media supplemented with 10% fetal bovine serum. Twenty-five female athymic nude mice (Harlan, Indianapolis, Ind.) were anaesthetized with isofluorane and inoculated s.c. in the right flank with 1.3×10⁶ PC-3 tumor cells suspended in 150 μL PBS. Tumor growth was monitored by weekly caliper measurement, and tumor volumes calculated as follows: (Width)²×Length/2. Mice were randomized into 4 groups of 7 based on their tumor volumes (150-300 mm³). Mice were given free access to food and water throughout the study. The mice were given potassium iodide at a concentration of 0.1% in their drinking water with the addition of 0.4% sweetener to aid palatability three days prior to injection and continuing through one week post injection in order to block thyroid uptake of possible free iodide.

Treatment:

The mice were injected with a 30 G ½ in. needle via lateral tail vein. Group 1 was injected with saline (150 μl per animal). Group 2 was injected with 1× Cold (vehicle) I-127-CLR1404, mass 25.33 μg/mL, volume 150 μL and 100 μCi I-131-CLR1404. Group 3 was injected with 10× Cold, 253.3 μg/ml, volume 150 μL and I-131-CLR1404, mass 25.9 μg/mL, radioactivity ˜97-120 μCi, volume 150 μL. Group 4 was injected with 100× Cold, I-127-CLR1401, 2533.3 μg/ml, volume 1504 and I-131-CLR1404, mass 25.9 μg/mL, radioactivity ˜97-120 μCi, volume 150 μL. The non-radioactive animals were housed in groups of 3-4 in cages in a separate rack from the radioactive animals. Radioactive animals were housed individually with lead shielding between cages.

Results:

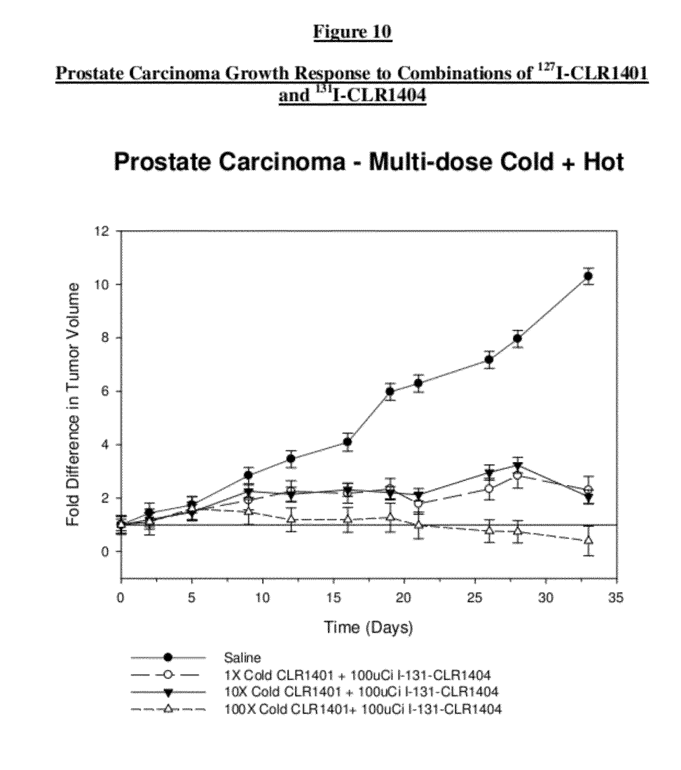

The over activation of the Akt/PI3K pathway is a known mediator of radiation resistance in cancer. Having shown that CLR1401 has significant cytotoxic properties that are selective for malignant cancer cell lines while sparing normal cells we next evaluated the effects on human tumor xenografts in vivo. Inhibition of Akt has been previously shown to sensitize cancer cells to the effects of radiation. Because the cold compound, CLR1401, has strong Akt inhibitory properties we combined multiple doses (4) of CLR1401 with a therapeutic dose of the radioactive compound ¹³¹I-CLR1404 in a prostate carcinoma (human PC-3 senograft) animal tumor model. Animals were given either 1×(3.8 μg), 10×(38 μg), or 100×(380 μg) of CLR1401 intravenously once a week for 4 weeks, saline was used as a control. One week after the first dose of CLR1401, animals were given a single dose of 100 μCi of I-131-CLR1404. High doses of CLR1401 had a striking effect on the tumor growth when used in combination with the radioactive compound (FIG. 10).

Not only did the combination therapy greatly inhibit tumor growth, it caused complete remission in 2 out of the 6 animals in the high dose (100×) group. One animal out of 6 in the mid-range treatment group also had complete remission. This is highly unusual in a subcutaneous xenograft cancer models. Typically, successful results are seen when a compound inhibits the growth of a tumor as compared to controls, rarely has there ever been observed complete tumor remission (no visible tumor) after treatment.

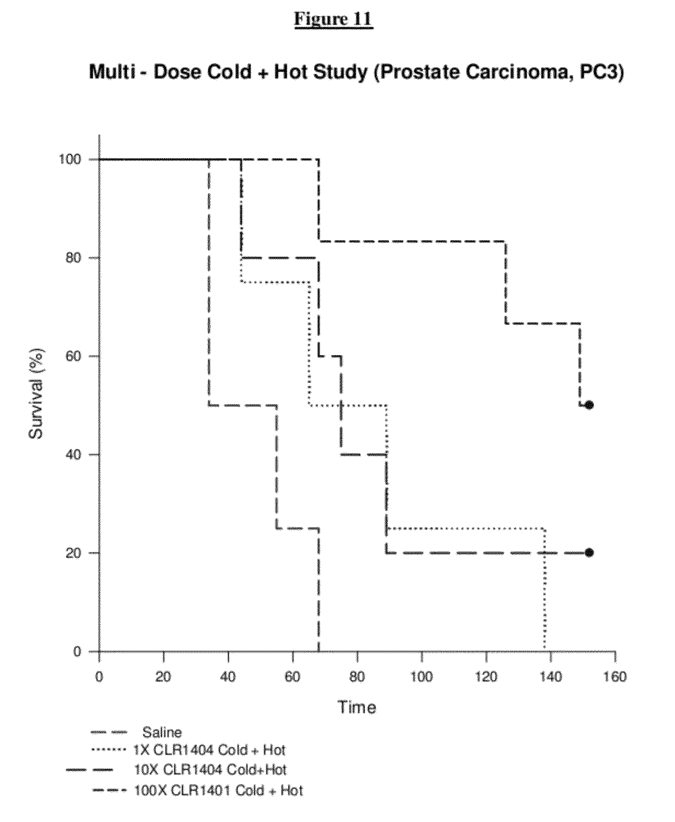

As would be expected from the tumor growth inhibition, there was also a significant and dramatic increase in the median survival time in the treatment groups (FIG. 11). The median survival time of the control (saline) treated group was 34 days, the median survival times of the 1×, 10×, and 100× treatment groups were 65, 75, and 149 days respectively. This increased survival time with the combination treatment of CLR1401 and ¹³¹I-CLR1404 is striking particularly given the average normal life-span of a mouse is only 500 days. The survival portion of this study is currently ongoing as there are animals in the 10× and 100× combination treatment groups that have not died. Because these animals no longer have tumors, and show no signs of metastatic disease the survival study could realistically continue throughout the course of their natural life.

### 5.9 Example 4

### ¹²⁷I-CLR1401 and ¹³¹I-CLR1404 are Effective in Treatment of Non-Small Cell Lung Cancer

Experimental Conditions:

The A549 cell line (human non-small lung cancer cell) was purchased from American Type Culture Collection (ATCC, Rockville, Md.) and maintained in F-12K media supplemented with 10% fetal bovine serum. Twenty-five female athymic nude mice (Harlan, Indianapolis, Ind.) were anaesthetized with isofluorene and inoculated s.c. in the right flank with 1.0×10⁶ A549 tumor cells suspended in 150 μL PBS. Tumor growth was monitored by weekly caliper measurement, and tumor volumes calculated as follows: (Width)²×Length/2. Mice were randomized into 4 groups of 7 based on their tumor volumes (150-300 mm³). Mice were given free access to food and water throughout the study. The mice were given potassium iodide at a concentration of 0.1% in their drinking water with the addition of 0.4% sweetener to aid palatability three days prior to injection and continuing through one week post injection in order to block thyroid uptake of possible free iodide.

Treatment:

The mice were injected with a 30 G ½ in. needle via lateral tail vein. Group 1 was injected with saline (150 μl per animal). Group 2 was injected with saline, volume 150 μL and 100 μCi I-131-CLR1404. Group 3 was injected with 30× Cold, 760 μg/ml, volume 1504 and I-131-CLR1404, mass 25.9 μg/mL, radioactivity ˜97-120 μCi, volume 1504. Group 4 was injected with 100× Cold, I-127-CLR1401, 2533.3 μg/ml, volume 1504 and I-131-CLR1404, mass 25.9 μg/mL, radioactivity ˜97-120 μCi, volume 150 μL. Group 5 was injected with 100× Cold I-127-CLR1401 only 2533.3 μg/ml, volume 150 μl. All animals received a total of 5 injections, one injection per week for five weeks. The non-radioactive animals were housed in groups of 3-4 in cages in a separate rack from the radioactive animals. Radioactive animals were housed individually with lead shielding between cages.

Results:

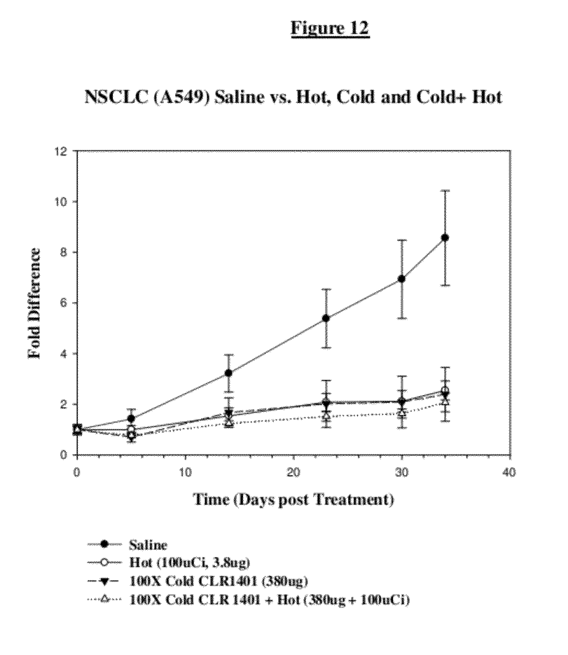

In the non-small cell lung cancer (NSCLC) model, A549, the cold compound, CLR1401, dramatically inhibits tumor growth in vivo. Mice bearing NSCLC (A549) tumors show a distinct tumor growth inhibition following treatment with 100×CLR1401 (380 μg per injection). This growth inhibition is statistically similar to the growth inhibition seen with the radioactive drug alone (FIG. 12). Unlike the “Cold”+“Hot” drug combination synergistic inhibition of tumor growth seen with the prostate carcinoma cell line, PC-3, no synergy was observed with the A549 model. This is most likely due to the genetic make up of the A549 cell line. This NSCLC cell line, has an intact PTEN, Akt, PI3K pathway, and does not express overactive levels of Akt activation. Therefore, it is less likely that a combination Akt inhibitor and cell selective radiation treatment would have a synergistic effect. This experiment is currently ongoing so there is currently no survival data available.

### 6.0 Example 5

### ¹²⁷I-CLR1401 and ¹³¹I-CLR1404 are Effective in Treatment of Triple Negative Breast Cancer

Experimental Conditions:

The MDA-MB-231 cell line (human mammary adenocarcinoma) was purchased from American Type Culture Collection (ATCC, Rockville, Md.) and maintained in Leibovitz's L-15 media supplemented with 10% Fetal Bovine Serum (FBS). Fifteen female athymic nude mice (Charles River, Portage, Mich.) were anesthetized with isofluorene and inoculated subcutaneously in the left flank with 3×10⁶ A549 cells suspended in 100 μL of PBS. Tumor growth was monitored weekly with caliper measurement. Tumor volume was calculated as follows: (Width)²×Length/2. Mice were randomized into 5 groups of 8 based on their volume (75-100 mm³). Mice were given free access to food and water throughout the study.

Treatment:

The mice were injected with 30 G ½ inch needle by tail vein injection. Group 1 (Saline) received 100 μL of saline for 5 weeks. Group 2 (Hot) received 100 μCi of I-131-CLR1404 on week 2 and the rest of the week, the animal received 100 μL of saline. Group 3 (Hot+100× Cold) received 100 μL of 100× cold (0.38 mg of I-127-CLR1404) on week 1,3,4 and 5 and 100 μCi of I-131-CLR1404 on week 2. Group 4 (100× Cold) received 100 μL of 100× cold (0.38 mg of I-127-CLR1404) for 5 weeks. Group 5 (Hot+30× Cold) received 100 μL of 30× cold (0.126 mg of I-127-CLR1404) on week 1,3,4 and 5 and 100 μCi of I-131-CLR1404 on week 2. The animals received 0.0004 mg/mL KI to block thyroid three days before hot injection and two weeks post hot injection except Group 4 which received 100× Cold injection.

Results:

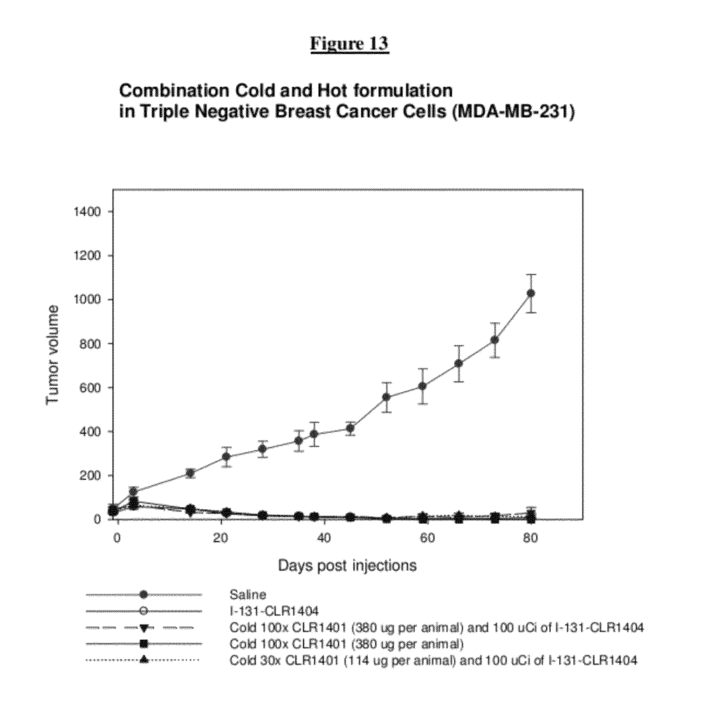

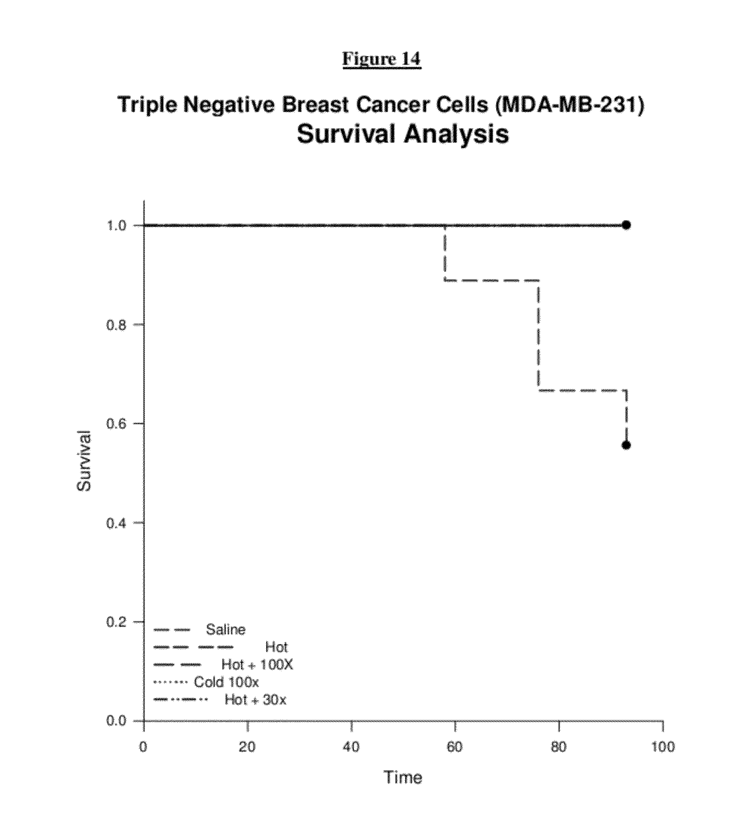

In the triple negative human mammary adenocarcinoma, MDA-MB-231 (which lacks of three receptors: estrogen receptors, progesterone receptors and human epidermal growth factor receptor (HER2)), the cold compound, CLR1401, dramatically inhibits tumor growth in vivo (P<0.001, Two repeated ANOVA, Sigma Plot 11) as seen in FIG. 13. The growth inhibition profile is similar to the growth seen in A549 tumor model. A549 and MDA-MB-231 share the same cell characteristics (has intact PTEN, Akt, PI3K pathway and does not express overactive levels of Akt activation). Mice bearing MDA-MB-231 tumors show a distinct tumor growth inhibition following cold treatment with 100×CLR1401 (380 μg per injection). The tumor inhibition of 100×CLR1401 has a similar therapeutic efficiency as hot treatment (I-131-CLR1404) or combination between hot and 30×CLR1404 or hot and 100×CLR1401. The Kaplan-Meier survival graph and log rank analysis shows survival benefit from all treatment groups (cold, hot or combination between hot and cold) as compared to saline (control) (P<0.001, Log rank, Sigma Plot 11) as seen in FIG. 14.

All treated mice were still alive after more than 90 days of the experiment and as of the filing date of this patent application.

### 6.1 Example 6

### Comparing the Efficacy of ¹²⁷I-CLR1401 Versus Erlotinib in the Treatment of Non-Small Cell Lung Cancer

Experimental Conditions:

The A549 cell line (human non small cell lung cancer) was purchased from American Type Culture Collection (ATCC, Rockville, Md.) and maintained in F-12K media supplemented with 10% Fetal Bovine Serum (FBS). Fifteen female athymic nude mice (Charles River, Portage, Mich.) were anesthetized with isofluorene and inoculated subcutaneously in the left flank with 1×10⁶ A549 cells suspended in 100 μL of PBS. Tumor growth was monitored weekly with caliper measurement. Tumor volume was calculated as follows: (Width)²×Length/2. Mice were randomized into 3 groups of 5 based on their volume (75-100 mm³). Mice were given free access to food and water throughout the study.

Treatment:

The mice were injected with 30 G ½ inch needle by tail vein injection for saline and cold groups weekly. Erlotinib group received 0.25 mg erlotinib per animal via intraperitonial daily for 3.5 weeks. Saline group received 100 μL of saline and cold group received 0.38 mg in 100 μL solution weekly for five weeks.

Results:

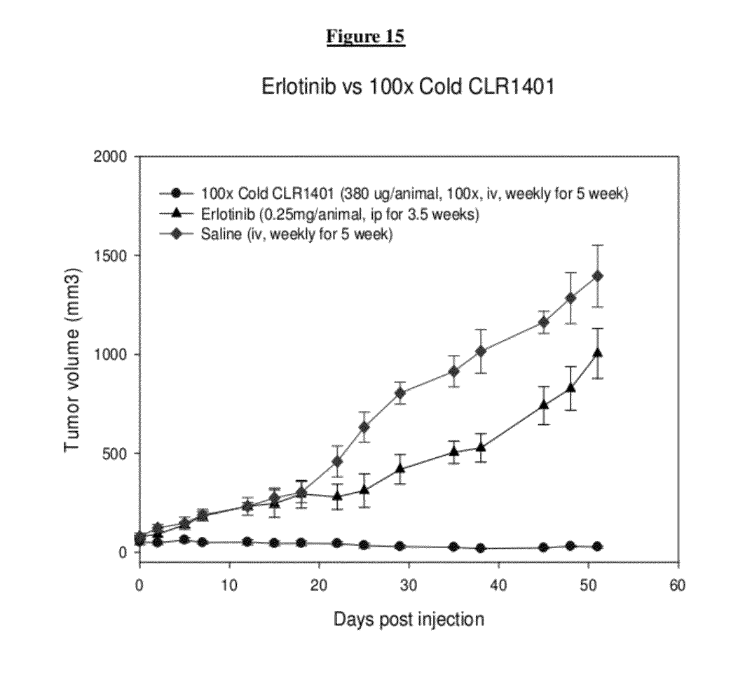

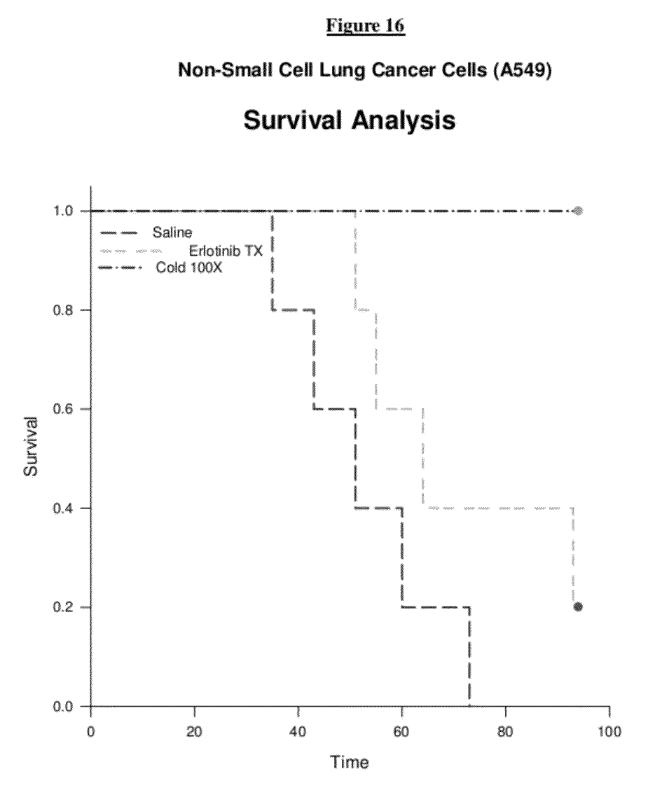

The “cold” molecule, 100×CLR1401 (0.38 mg per animal), significantly inhibited tumor growth in Non-Small Cell Lung Cancer (NSCLC) model as compared to saline (control) or 0.25 mg Erlotinib as shown in FIG. 15 (P<0.001, Two Way Repeated ANOVA, Sigma Plot 11). Erlotinib is designed to block tumor cell growth by targeting the epidermal growth factor receptor (HER1/EGFR). Erlotinib is commonly used as monotherapy or combined therapy for patient with advanced NSCLC. On the other hand, as previously discussed above, CLR1401 was shown to be inhibiting Akt activation. The experiment has demonstrated that the I-127-CLR1404 treatment is superior to monotherapy of Erlotinib. The Kaplan Meier survival graph and log rank analysis shows survival benefit from cold compound as compared to saline or erlotinib (P=0.002, Log rank, Sigma Plot 11) as seen in FIG. 16.

All publications and patent applications cited in this specification are herein incorporated by reference as if each individual publication or patent application were specifically and individually indicated to be incorporated by reference. Although the foregoing has been described in some detail by way of illustration and example for purposes of clarity of understanding, it will be readily apparent to those of ordinary skill in the art in light of the teachings of the specification that certain changes and modifications may be made thereto without departing from the spirit or scope of the appended claims.

1. A method of treating a solid cancer comprising administering to a patient in need thereof a therapeutically effective amount of a nonradioactive phospholipid compound selected from: 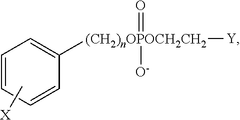
where X is: a) a nonradioactive isotope of iodine or b) H; n is an integer between 12 and 30; and Y is selected from the group comprising N⁺H₃, HN⁺(R)₂, N⁺H₂R, and N⁺(R)₃, wherein R is an alkyl or arylalkyl substituent and 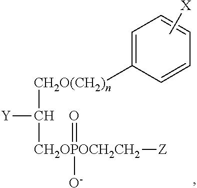
where X is: a) a nonradioactive isotope of iodine or b) H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent or a pharmaceutically acceptable salt thereof.

2. The method of claim 1, wherein the nonradioactive phospholipid compound is selected from the group consisting of 18-(p-Iodophenyl)octadecyl phosphocholine, 1-O-[8-(p-Iodophenyl)octadecyl]-1,3-propanediol-3-phosphocholine, and 1-O-[8-(p-Iodophenyl)octadecyl]-2-O-methyl-rac-glycero-3-phosphocholine, wherein iodine is a nonradioactive isotope.

3. The method of claim 1, wherein the nonradioactive phospholipid compound is of the formula: 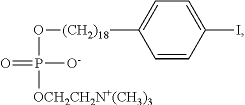
wherein I is a nonradioactive isotope of iodine, or a pharmaceutically acceptable salt thereof.

4. The method of claim 1, wherein said solid cancer is selected from the group consisting of lung cancer, breast cancer, glioma, squamous cell carcinoma, prostate cancer, melanoma, renal cancer, colorectal cancer, ovarian cancer, pancreatic cancer, sarcoma, and stomach cancer.

5. A combination pharmaceutical agent comprising a radioactive phospholipid compound selected from: 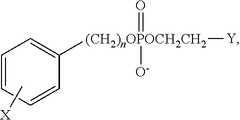
where X is a radioactive isotope of iodine; n is an integer between 12 and 30; and Y is selected from the group consisting of N⁺H₃, HN⁺(R)₂, N⁺H₂R, and N⁺(R)₃, wherein R is an alkyl or arylalkyl substituent or 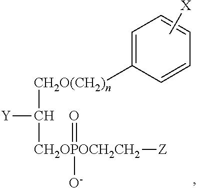
where X is a radioactive isotope of iodine; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent and a protein kinase B (Akt) inhibitor.

6. The combination pharmaceutical agent of claim 5, wherein said Akt inhibitor is a nonradioactive phospholipid compound selected from: 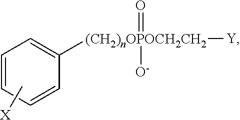
where X is: a) a nonradioactive isotope of iodine or b) H; n is an integer between 12 and 30; and Y is selected from the group consisting of N⁺H₃, HN⁺(R)₂, N⁺H₂R, and N⁺(R)₃, wherein R is an alkyl or arylalkyl substituent and 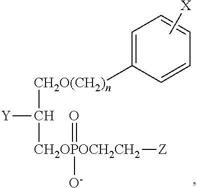
where X is: a) a nonradioactive isotope of iodine or b) H; n is an integer between 12 and 30; Y is selected from the group consisting of H, OH, COOH, COOR and OR, and Z is selected from the group consisting of N⁺H₃, HN⁺(R)₂, N⁺H₂R, and N⁺(R)₃, wherein R is an alkyl or arylalkyl substituent.

7. The combination pharmaceutical agent of claim 6, wherein the radioactive isotope of iodine is selected from the group consisting of ¹²³I, ¹²⁴I, ¹²⁵I, and ¹³¹I.

8. The combination pharmaceutical agent of claim 6, wherein said radioactive phospholipid compound is selected from the group consisting of 18-(p-Iodophenyl)octadecyl phosphocholine, 1-O-[18-(p-Iodophenyl)octadecyl]-1,3-propanediol-3-phosphocholine, and 1-O-[18-(p-Iodophenyl)octadecyl]-2-O-methyl-rac-glycero-3-phosphocholine, wherein iodine is a radioactive isotope.

9. A combination pharmaceutical agent comprising a nonradioactive phospholipid compound of the formula: 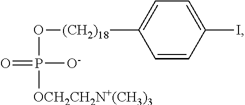
or a pharmaceutically acceptable salt thereof, wherein I is a nonradioactive isotope of iodine, and a radioactive phospholipid compound of the formula: 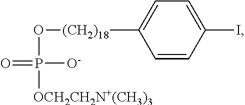
wherein I is a radioactive isotope of iodine.

10. The combination pharmaceutical agent of claim 9, wherein said radioactive isotope of iodine is selected from the group consisting of ¹²⁵I and ¹³¹I.

11. The combination pharmaceutical agent of claim 6, wherein said radioactive phosholipid compound and said nonradioactive compound are formulated as a single composition.

12. The combination pharmaceutical agent of claim 11, wherein the ratio of the nonradioactive phospholipid compound to the radioactive phospholipid compound is about 10:1 by weight.

13. The combination pharmaceutical agent of claim 6, wherein said radioactive phosholipid compound and said nonradioactive compound are formulated as separate compositions.

14. The combination pharmaceutical agent of claim 6, wherein said combination pharmaceutical agent allows to reach the serum concentration of said nonradioactive compound of between about 5 μM and about 10 μM when administered to a human patient.

15. A method of treating a solid cancer comprising administering to a patient in need thereof a therapeutically effective amount of the combination pharmaceutical agent of claim 5.

16. The method of claim 15, wherein said solid cancer is selected from the group consisting of lung cancer, breast cancer, glioma, squamous cell carcinoma, prostate cancer, melanoma, renal cancer, colorectal cancer, ovarian cancer, pancreatic cancer, sarcoma, and stomach cancer.

17. The method of claim 15, wherein said therapeutically effective amount is from about 7 mCi to about 700 mCi.

18. A method of treating a solid cancer comprising administering to a patient in need thereof a therapeutically effective amount of the combination pharmaceutical agent of claim 9.
